(19) United States
(12) Patent Application Publication
(10) Pub. No.: US 2012/0114638 A1
(43) Pub. Date: May 10, 2012

(54) COMBINATION THERAPY

(76) Inventors: John Boylan, San Diego, CA (US); Stanislaw Mikulski, Essex Fells, NJ (US)

(21) Appl. No.: 13/279,367

(22) Filed: Oct. 24, 2011

Related U.S. Application Data
(60) Provisional application No. 61/410,960, filed on Nov. 8, 2010.

(51) Int. Cl. A61K 39/395; A61P 35/00

(52) U.S. Cl. 424/133.1

(57) ABSTRACT

The present invention relates to a combination therapy of 2,2-dimethyl-N-((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, and bevacizumab for treating a patient suffering from a proliferative disorder, in particular a solid tumor, for example a brain tumor.

### PRIORITY TO RELATED APPLICATION(S)

This application claims the benefit of U.S. Provisional Application No. 61/410,960, filed Nov. 8, 2010. The entire contents of the above-identified application is hereby incorporated by reference.

### FIELD OF THE INVENTION

The present invention relates to a combination therapy for treating a patient suffering from a proliferative disorder.

### BACKGROUND OF THE INVENTION

Bevacizumab is an anti-VEGF monoclonal antibody which has demonstrated utility in the treatment of infiltrating gliomas. However, the long term use of such antibodies may be limited due to adoption of the invasive phenotype by tumor cells.

2,2-Dimethyl-N—((S)-6-oxo-6,7-dihydro-5H-dibenzo[b,d]azepin-7-yl)-N′-(2,2,3,3,3-pentafluoro-propyl)-malonamide is disclosed in WO 2005/023772 as useful for the treatment of Alzheimer's disease. It is also known to be a potent and selective inhibitor of γ-secretase, a key enzyme responsible for the cleavage and activation of Notch receptors. The Notch pathway is known to have a role in mediating invasion. Further, dysregulation of Notch signaling due to gene amplification, chromosomal translocation, or mutations has been implicated in many types of cancers including leukemia, medullo- and glioblastoma, breast carcinoma, head and neck cancer, and pancreatic carcinoma. Preclinical evidence has shown that blockade of Notch signaling through inhibition of the proteolytic activity of γ-secretase results in deterring tumor growth in mouse xenograft models.

### SUMMARY OF THE INVENTION

The present invention provides a method of treating a patient suffering from a proliferative disorder, comprising administering to the patient: (A) a first component which comprises, as an active agent, 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof; and (B) a second component which comprises, as an active agent, bevacizumab; the amounts of said active agents being such that the combination thereof is therapeutically-effective in the treatment of said proliferative disorder.

The present invention also provides a kit comprising: (A) a first component which comprises, as an active agent, 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof; and (B) a second component which comprises, as an active agent, bevacizumab.

The present invention further provides a composition comprising: (A) a first component which comprises, as an active agent, 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof; and (B) a second component which comprises, as an active agent, bevacizumab.

In addition, the present invention provides a use of 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, and bevacizumab for the treatment of a proliferative disorder.

The present invention further provides a use of 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, and bevacizumab for the preparation of a medicament for the treatment of a proliferative disorder.

### DETAILED DESCRIPTION OF THE INVENTION

As used herein, the following terms have the meanings set out below.

As used herein, the term “Compound I” shall refer to 2,2-dimethyl-N—((S)-6-oxo-6,7-dihydro-5H-dibenzo[b,d]azepin-7-yl)-N′-(2,2,3,3,3-pentafluoro-propyl)-malonamide. The compound has the structure shown below in formula (I),

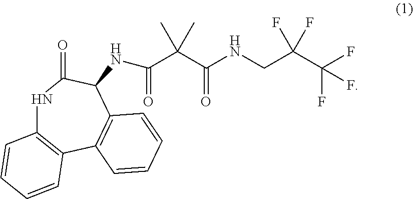

The term “antineoplastic” means inhibiting or preventing the development, maturation or proliferation of malignant cells.

The term “area under the curve” (AUC) is the area under the curve in a plot of concentration of drug in plasma against time. AUC represents the total amount of drug absorbed by the body, irrespective of the rate of absorption. This is useful for the therapeutic monitoring of drugs. Measurement of the drug concentrations in a patient's plasma and calculation of the AUC is useful to guide the dosage of this drug. AUC becomes useful for knowing the average concentration over a time interval, AUC/t. AUC is generally expressed as (mass*time/volume), for example, ng-hr/ml.

The term “pharmaceutically acceptable,” such as pharmaceutically acceptable carrier, excipient, etc., means pharmacologically acceptable and substantially non-toxic to the subject to which the particular compound is administered. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. The term “pharmaceutically acceptable ester” of a compound means a conventionally esterified compound having a carboxyl group, which esters retain the biological effectiveness and properties of the compound. Chemical modification of a pharmaceutical compound (i.e., drug) into a salt is a technique well known to pharmaceutical chemists to obtain improved physical and chemical stability, hydroscopicity, and solubility of compounds. See, e.g., H. Ansel et. al., Pharmaceutical Dosage Forms and Drug Delivery Systems (6^{th} Ed. 1995) at pp. 196 and 1456-1457.

The term “prodrug” refers to compounds, which undergo transformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

The term “therapeutically-effective amount” means an amount of drug, which is effective for producing a desired therapeutic effect upon administration to a patient, for example, to stem the growth, or result in the shrinkage, of a cancerous tumor.

The term “therapeutic index” is an important parameter in the selection of anticancer agents for clinical trial. Therapeutic Index takes into consideration the efficacy, pharmacokinetecs, metabolism and bioavailability of anticancer agents. See, e.g., J. Natl. Cancer Inst. 81(13): 988-94 (Jul. 5, 1989).

The term “tumor control” means that the perpendicular diameters of measurable lesions have not increased by 25% or more from the last measurement. See, e.g., World Health Organization (“WHO”) Handbook for Reporting Results of Cancer Treatment, Geneva (1979).

The present invention relates to a method of treating a patient suffering from a proliferative disorder, comprising administering to the patient: (A) a first component which comprises, as an active agent, Compound I, or a pharmaceutically-acceptable salt thereof; and (B) a second component which comprises, as an active agent, bevacizumab; the amounts of said active agents being such that the combination thereof is therapeutically-effective in the treatment of said proliferative disorder.

Treatment of a proliferative disorder shall be understood to include maintaining or decreasing tumor size, inducing tumor regression (either partial or complete), inhibiting tumor growth, and/or increasing the life span of a patient suffering from said disorder.

In an embodiment of the present invention, the patient is a human.

In an embodiment of the invention, the proliferative disorder is a solid tumor, for example a brain tumor. In an embodiment of the invention, the disorder is a glioma, for example, a malignant glioma. Examples of such disorders include glioblastoma, anaplastic astrocytoma, anaplastic oligodendroglioma, and mixed anaplastic oligoastrocytoma.

As stated above, each of the active agents administered in the aforementioned method are administered in amounts such that the combination thereof is therapeutically-effective in the treatment of said proliferative disorder.

The dosages may be administered according to any dosage schedule determined by the physician in accordance with the requirements of the patient. For example, the dosages of each of the two components may be administered in single or in divided doses over a period of several days, or alternating daily schedules.

In an embodiment, Compound I, or a pharmaceutically-acceptable salt thereof is administered in a treatment schedule that is repeated every twenty eight days (a 28 day cycle), every twenty one days (a 21 day cycle), or as soon as permitted by recovery from toxicity, for so long as the tumor is under control and the patient tolerates the regiment or tumor regression. In an embodiment, these treatment cycles are repeated for a total of up to about eight cycles.

In an embodiment, Compound I, or a pharmaceutically-acceptable salt thereof, is administered once daily on days 1, 2, 3, 8, 9, 10, 15, 16, 17, 22, 23, and 24 of a 28 day cycle. In another embodiment, Compound I, or a pharmaceutically-acceptable salt thereof, is administered once daily on days 1, 2, 3, 8, 9, and 10 of a 21 day cycle. In yet another embodiment, Compound I, or a pharmaceutically-acceptable salt thereof, is administered once daily on days 1-7 of a 21 day cycle. In yet another embodiment, Compound I, or a pharmaceutically-acceptable salt thereof, is administered once daily on days 1, 3, 5, 7, 9, 11, 13, 15, 17, 19, and 21 of a 21 day cycle. In yet another embodiment, Compound I, or a pharmaceutically-acceptable salt thereof, is administered once daily on days 1, 8, and 15 of a 21 day schedule. In yet another embodiment, Compound I, or a pharmaceutically-acceptable salt thereof, is administered once daily on days 1, 4, 8, 11, 15, and 18 of a 21 day schedule. In yet another embodiment, Compound I, or a pharmaceutically-acceptable salt thereof, is administered once daily on days 1, 2, 3, 4, 5, 8, 9, 10, 11, 12, 15, 16, 17, 18, and 19 of a 21 day schedule.

In an embodiment, each administration of Compound I, or a pharmaceutically-acceptable salt thereof, is in an amount of from about 1 mg to about 100 mg, from about 1 mg to about 50 mg, from about 1 mg to about 25 mg, or from about 5 mg to about 20 mg.

In an embodiment, bevacizumab is administered one every three weeks. In another embodiment, bevacizumab is administered once every two weeks.

In an embodiment, each administration of bevacizumab is in an amount of from about 1 μg/kg to about 100 mg/kg, from about 1 μg/kg to about 50 mg/kg, from about 0.1 mg/kg to about 20 mg/kg, from 1 mg/kg to about 20 mg/kg, from about 5 mg/kg to about 15 mg/kg, or about 10 mg/kg.

In an embodiment, bevacizumab is administered once every two weeks in an amount of about 3 mg/kg. In a further embodiment, bevacizumab is administered once every two weeks in an amount of about 5 mg/kg. In another embodiment, bevacizumab is administered once every two weeks in an amount of about 10 mg/kg. In yet another embodiment, bevacizumab is administered once every three weeks in an amount of about 15 mg/kg.

The dosage levels of each of the components may be modified by a physician to be lower or higher than that stated herein depending on the needs of the patient, and the reaction of the patient to the treatment.

In an embodiment of the present invention, Compound I, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 100 mg and bevacizumab is administered once every two weeks (for example, on days 1 (+2 days) and 15 (±2 days)) in an amount of from about 5 mg/kg to about 15 mg/kg.

In another embodiment of the present invention, Compound I, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 50 mg and bevacizumab is administered once every two weeks (for example, on days 1 (+2 days) and 15 (±2 days)) in an amount of from about 5 mg/kg to about 15 mg/kg.

In another embodiment of the present invention, Compound I, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 25 mg and bevacizumab is administered once every two weeks (for example, on days 1 (+2 days) and 15 (±2 days)) in an amount of from about 5 mg/kg to about 15 mg/kg.

In another embodiment of the present invention, Compound I, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 25 mg and bevacizumab is administered once every two weeks (for example, on days 1 (+2 days) and 15 (±2 days)) in an amount of about 10 mg/kg.

In another embodiment of the present invention, Compound I, or a pharmaceutically-acceptable salt thereof, is administered once daily on days 1, 2, 3, 8, 9, 10, 15, 16, 17, 22, 23, and 24 of a 28 day cycle in an amount of from about 5 mg to about 20 mg and bevacizumab is administered once every two weeks (for example, on days 1 (+2 days) and 15 (±2 days)) in an amount of from about 5 mg/kg to about 15 mg/kg.

In another embodiment of the present invention, Compound I, or a pharmaceutically-acceptable salt thereof, is administered once daily on days 1, 2, 3, 8, 9, 10, 15, 16, 17, 22, 23, and 24 of a 28 day cycle in an amount of from about 5 mg to about 20 mg and bevacizumab is administered once every two weeks (for example, on days 1 (+2 days) and 15 (±2 days)) in an amount of about 10 mg/kg.

In an embodiment, Compound I, or a pharmaceutically-acceptable salt thereof, is in a pharmaceutical oral unit dosage form, for example a tablet. In an embodiment, the tablet may be a 1 mg, a 10 mg or a 20 mg tablet.

In an embodiment, bevacizumab is administered as an infusion. The infusion may be, for example, over the course of about 30 minutes, about 60 minutes, or about 90 minutes.

Treatment with either agent may be sustained until a desired suppression of disease symptoms occurs or as long as the cancer remains under control.

The present invention also relates to the use of 2,2-dimethyl-N—((S)-6-oxo-6,7-dihydro-5H-dibenzo[b,d]azepin-7-yl)-N′-(2,2,3,3,3-pentafluoro-propyl]-malonamide, or a pharmaceutically-acceptable salt thereof, and bevacizumab for the treatment of a proliferative disorder.

The present invention further relates to the use of 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, and bevacizumab for the preparation of a medicament for the treatment of a proliferative disorder.

### EXAMPLES

### Example 1

Over a four week (28 day) cycle, patients receive 2,2-dimethyl-N—((S)-6-oxo-6,7-dihydro-5H-dibenzo[b,d]azepin-7-yl)-N′-(2,2,3,3,3-pentafluoro-propyl)-malonamide orally on days 1-3, 8-10, 15-17, and 22-24 at 5 mg, 10 mg, or 20 mg per dose and bevacizumab at 10 mg/kg on days 1 (+2 days) and 15 (±2 days) by intravenous infusion. The first dose of bevacizumab is given over 90 minutes. If well tolerated, the second dose is given over 60 minutes. If this dose is well-tolerated, then all subsequent infusions of bevacizumab (e.g., in further cycles) can be administered over 30 minutes.

The treatment cycle is repeated until disease progression or unit development of significant toxicities.

1. A method of treating a patient suffering from a proliferative disorder, comprising administering to the patient: (A) a first component which comprises, as an active agent, 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof; and (B) a second component which comprises, as an active agent, bevacizumab; the amounts of said active agents being such that the combination thereof is therapeutically-effective in the treatment of said proliferative disorder.

2. A method according to claim 1 wherein said patient is a human.

3. A method according to claim 1 wherein said proliferative disorder is a solid tumor.

4. A method according to claim 1 wherein said proliferative disorder is a brain tumor.

5. A method according to claim 1 wherein said proliferative disorder is a glioma.

6. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 100 mg.

7. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 50 mg.

8. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on days 1, 2, 3, 8, 9, 10, 15, 16, 17, 22, 23, and 24 of a 28 day cycle in an amount of from about 1 mg to about 25 mg.

9. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof, is administered once daily on days 1, 2, 3, 8, 9, 10, 15, 16, 17, 22, 23, and 24 of a 28 day cycle in an amount of from about 5 mg to about 20 mg.

10. A method according to claim 1 wherein bevacizumab is administered in an amount of from about 5 mg/kg to about 15 mg/kg once every two weeks.

11. A method according to claim 1 wherein bevacizumab is administered in an amount of about 10 mg/kg once every two weeks.

12. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof is administered in an oral dosage form.

13. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof is administered in the form of a tablet.

14. A method according to claim 1 wherein bevacizumab is administered in the form of an infusion.

15. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof is administered once daily on days 1, 2, 3, 8, 9, 10, 15, 16, 17, 22, 23, and 24 of a 28 day cycle in an amount of from about 1 mg to about 25 mg and bevacizumab is administered in an amount of from about 5 mg/kg to about 15 mg/kg once every two weeks.

16. A method according to claim 1 wherein 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof is administered once daily on days 1, 2, 3, 8, 9, 10, 15, 16, 17, 22, 23, and 24 of a 28 day cycle in an amount of from about 5 mg to about 20 mg and bevacizumab is administered in an amount of about 10 mg/kg once every two weeks.

17. A kit comprising: (A) a first component which comprises, as an active agent, 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof; and (B) a second component which comprises, as an active agent, a bevacizumab.

18. A composition comprising: (A) a first component which comprises, as an active agent, 2,2-dimethyl-N—((S)-6-oxo-6,7-dihydro-5H-dibenzo[b,d]azepin-7-yl)-N′-(2,2,3,3,3-pentafluoro-propyl)-malonamide, or a pharmaceutically-acceptable salt thereof; and (B) a second component which comprises, as an active agent, a bevacizumab.
